(19) United States
(12) Patent Application Publication
(10) Pub. No.: US 2002/0198589 A1
(43) Pub. Date: Dec. 26, 2002

(54) Tessellated stent and method of manufacture

(76) Inventor: Veronica Jade Leong, Ithaca, NY (US)

Correspondence Address: FULWIDER PATTON LEE & UTECHT, LLP, Attorneys at Law, Howard Hughes Center, 6060 Center Drive, Tenth Floor, Los Angeles, CA, 90045, US

(21) Appl. No.: 09/888,091

(22) Filed: Jun. 22, 2001

(51) Int. Cl. A61F 2/06

(52) U.S. Cl. 623/1.15

(57) ABSTRACT

An improved tessellated stent is cut from a single length of tubing. The stent consists of a plurality of expandable cylindrical rings aligned on a common axis and connected by links. The rings having a parallelogram but non-rectangular cross-section. The stent is manufactured by direct laser cutting. A laser beam is focused so it is not radially aligned through the stent's center.

### BACKGROUND OF THE INVENTION

This invention relates generally to improvements of expandable stents and their manufacture. More particularly, it relates to a stent that crimps into a lower profile and to methods of manufacture of that stent.

Stents are expandable endoprostheses which are implanted into a body lumen, such as a blood vessel, to maintain the patency of the vessel. These devices are typically used in the treatment of atherosclerotic stenosis in blood vessels, most notably in the coronary arteries, but elsewhere as well. They are also useful to support and hold back a dissected arterial lining which can occlude the fluid passageway. The stents themselves are typically thin-walled, expandable tubes. Further details of stents can be found in U.S. Pat. No. 3,868,956 (Alfidi et al.); U.S. Pat. No. 4,512,338 (Balko et al.); U.S. Pat. No. 4,553,545 (Maass et al.); U.S. Pat. No. 4,733,665 (Palmaz); U.S. Pat. No. 4,762,128 (Rosenbluth); U.S. Pat. No. 4,800,882 (Gianturco); U.S. Pat. No. 4,856,516 (Hillstead); U.S. Pat. No. 4,886,062 (Wiktor); U.S. Pat. No. 5,421,955 (Lau); and U.S. Pat. No. 5,569,295 (Lam), which are hereby incorporated herein in their entirety by reference thereto.

Various means have been provided to deliver and implant stents. One method frequently described for delivering a stent includes: mounting the expandable stent on an expandable member, such as an angioplasty balloon on the distal end of an intravascular catheter; advancing the catheter to the desired location within the patient's body lumen; inflating the balloon on the catheter to expand the stent into a permanent expanded condition; and, then deflating the balloon and removing the catheter. Further details of dilatation catheters, guide wires, and devices associated therewith for angioplasty procedures can be found in U.S. Pat. No. 4,323,071 (Simpson-Robert); U.S. Pat. No. 4,439,185 (Lindquist); U.S. Pat. No. 4,516,972 (Samson); U.S. Pat. No. 4,538,622 (Samson, et al.); U.S. Pat. No. 4,554,929 (Samson, et al.); U.S. Pat. No. 4,616,652 (Simpson); U.S. Pat. No. 4,638,805 (Powell); U.S. Pat. No. 4,748,982 (Horzewski, et al.); U.S. Pat. No. 5,507,768 (Lau, et al.); U.S. Pat. No. 5,451,233 (Yock); and U.S. Pat. No. 5,458,651 (Klemm, et al.), which are hereby incorporated herein in their entirety by reference thereto.

One example of a particularly useful stent is the MultiLink® family of stents manufactured by Advanced Cardiovascular Systems, Inc., Santa Clara, Calif. (“ACS”). That family includes the Duet®, Penta® and Tetra® stents, and others. These stents are relatively flexible along their longitudinal axis to facilitate delivery through tortuous body lumens, but they are stiff and stable enough radially in an expanded condition to maintain the patency of a body lumen. Each stent typically includes a plurality of radially expandable cylindrical elements, or rings, which are relatively independent of each other in their ability to expand and to flex. The individual, radially expandable, cylindrical elements of the stent are precisely dimensioned so as to be longitudinally shorter than their own diameters. Interconnecting elements, also known as links, extend between adjacent cylindrical elements. They provide increased stability and help prevent warping of the stent as it is expanded. The resulting stent structure is a series of radially expandable, cylindrical elements which are spaced longitudinally close enough so that small stenoses or dissections in the wall of a body lumen may be pressed back into position against the luminal wall, but not so close as to compromise the longitudinal flexibility of the stent. The individual cylindrical elements may rotate slightly relative to adjacent cylindrical elements without significant deformation, cumulatively giving a stent which is flexible along its length and about its longitudinal axis, but is still very stiff in the radial direction in order to resist collapse.

This family of stents has a precise, circumferentially undulating or serpentine pattern. The transverse cross-section of the cylindrical element is almost rectangular and preferably has an aspect ratio of about two to one to about 0.5 to one. Some in the art view a one to one aspect ratio as particularly suitable. The open, reticulated structure of the stent allows for the perfusion of blood over a large portion of the arterial wall, which can improve the healing and repair of a damaged arterial lining. A method for making such stents is disclosed in U.S. Pat. Nos. 5,759,192 and 5,780,807 to Saunders. These two patents are incorporated herein by reference in their entirety.

The radial expansion of the expandable cylinder deforms the undulating pattern similar to changes in a waveform which result from decreasing the waveform's amplitude and the frequency. Preferably, the undulating patterns of the individual cylindrical structures are in phase with each other in order to prevent the contraction of the stent along its length when it is expanded. The expansion plastically deforms the stent so it will remain in the expanded condition. During expansion of the stent, portions of the undulating pattern may tip outwardly, resulting in projecting members on the outer surface of the expanded stent.

The links interconnecting adjacent cylindrical elements have a precisely defined, rectangular transverse cross-section similar to the cross-section of the expandable cylindrical elements, also simply known as rings. The links may be formed with the rings as a unitary structure from an intermediate product, such as a tube.

The number and location of links interconnecting adjacent cylindrical rings can be varied in order to develop the desired longitudinal flexibility in the stent structure both in the unexpanded, as well as the expanded condition. Generally, the greater the longitudinal flexibility of the stent, the more easily and safely it can be delivered to the implantation site. The same is true for the stent profile as it is crimped onto the balloon. The lower the profile is, the easier and safer the stent delivery is. This feature is becoming more important as interventional cardiologists implant stents without sheaths and without having performed prior angioplasty. When dealing with vessels the size of coronary arteries, an extra margin of a thousandth or ten thousandth of an inch is a feature interventional cardiologists look for.

Stents are very high precision, relatively fragile devices and, ideally, the most desirable metal stents possess a precision structure cut from a very small diameter, thin-walled, stainless steel cylindrical tube. It is extremely important to make precisely dimensioned, smooth, narrow cuts in the tubes in extremely fine geometries without damaging the narrow struts that make up the stent structure. While various prior art cutting processes, such as chemical etching, and electrical discharge machining (EDM) were previously deemed adequate, stent designers continually seek manufacturing techniques that result in stents with enhanced structural quality in terms of fluoroscopic resolution, reliable use, low profile and high flexibility.

Accordingly, those concerned with the development, manufacture and use of stents have long recognized the need for stents with even smaller profiles and for improved manufacturing processes for such stents. The present invention fulfills these needs.

### SUMMARY OF THE INVENTION

The present invention is a new design for an endovascular prosthesis such as a stent and a method for laser cutting the prosthesis. The prosthesis is preferably a stent with struts and links that form an expandable, tube-shaped lattice. Preferably, the stent is a series of cylindrical rings comprised of struts, with links connecting the rings. Rather than having a traditional, rectangular cross-section, the struts have parallel edges that are not colinear with the tube's radius. Prior art stents cut from tubes have struts with virtually rectangular cross-sections formed from cuts through the stent material that if extended, would pass through the center of the stent. In other words, the cuts, such as those made by a laser, would be colinear with the radius of the stent. In the present invention those cuts, if extended, would not pass through the stent's center. Rather, they would all be in the same direction, forming an angle to the stent's radius. Taking a cross section of one of the stent struts of the present invention would result in a parallelogram with obtuse and acute angles. When crimped, the edges of one strut can abut along the length of the edge of another strut, forming a tessellated surface on the outside of the crimped stent.

The tubes are typically made of stainless steel and cut by laser. They are placed on a fixture under a laser. A CNC machine is used to generate a very intricate and precise pattern. Due to the thin wall and the small geometry of the stent pattern, it is necessary to have very precise control of the laser, its power level, the focus spot size, and the precise positioning of the laser cutting path.

In addition to the laser and the CNC positioning equipment, the optical delivery system includes a beam expander to increase the laser beam diameter, a circular polarizer to eliminate polarization effects in metal cutting, provisions for a spatial filter, a binocular viewing head and focusing lens, and a coaxial gas jet, or jet stream, that provides for the introduction of a gas stream that surrounds the focused beam and is directed along the beam axis. The coaxial jet, pressurized with oxygen, is directed at the tube with the focused laser beam exiting the nozzle tip. The focused laser beam acts as an ignition source and controls the reaction of the oxygen with the metal. In order to prevent burning by the beam and/or molten slag on the far wall of the tube inside diameter, a stainless steel mandrel is placed inside the tube and is allowed to roll on the bottom of the tube as the pattern is cut. This acts as a beam/debris block protecting the far wall inside diameter.

The above and other objects and advantages of this invention will be apparent from the following more detailed description when taken in conjunction with the accompanying drawings of exemplary embodiments.

### DESCRIPTION OF THE DRAWINGS

FIG. 1 is an elevational view, partially in section, of a stent embodying features of the invention which is mounted on a delivery catheter and disposed within a damaged artery.

FIG. 2 is an elevational view, partially in section, wherein the stent is expanded within a damaged artery, pressing the damaged lining against the arterial wall.

FIG. 3 is an elevational view, partially in section, showing the expanded stent within the artery after withdrawal of the delivery catheter;

FIG. 4 is a perspective view of a stent with one end of the stent being shown in an exploded view to illustrate the details thereof.

FIG. 5 is a plan view of a flattened section of a stent of the invention which illustrates the undulating pattern of the stent shown in FIG. 4.

FIG. 6 is a sectional view taken along the line 6-6 in FIG. 5.

FIG. 7 is an exaggerated version of FIG. 6, showing that the cross-sectional shape of FIG. 6 is sometimes not precisely rectangular.

FIG. 8 is a stent cross-section, taken along line 8-8 in FIG. 4 depicting the unused crimping space in prior art stents.

FIG. 9 is a cross-section of the stent strut of the present invention, with the edges cut in the same direction.

FIG. 10 is a stent cross-section, also taken along line 8-8 in FIG. 4, showing a crimped stent with struts that can form a tessellated surface.

FIG. 11 is a cross-section of another embodiment of the invention.

FIG. 12 is a schematic representation of equipment for cutting the tubing in the manufacture of stents, in accordance with the present invention.

FIG. 13 is an elevational view of a system for cutting an appropriate pattern by laser in a metal tube to form a stent, in accordance with the invention.

FIG. 14 is a plan view of the laser head and optical delivery subsystem for the laser cutting system.

FIG. 15 is an elevational view of a coaxial gas jet, rotary collet, and bushing for use in the system of FIG. 13.

FIG. 16 is an elevational and schematic drawing of laser beam diameter vs. spot size and depth of focus.

FIG. 17 is an elevational and schematic drawing of focal length vs. spot size and depth of focus.

FIG. 18 is a plan of an alternative stent pattern that can be used with the present invention.

### DETAILED DESCRIPTION OF THE PREFERRED EMBODIMENTS

The present invention relates to the cross-sectional shape of endoluminal prostheses such a stents. It also involves a method for making such prostheses. Preferably, a laser is used to cut a metal hypotube, and the beam of the laser is offset from the center of the tube. In such a manner, a prosthesis such as a stent is cut to have struts with cross-sections of non-rectangular parallelograms.

Referring first to the drawings for a more general understanding, FIG. 1 shows an endovascular prosthesis such as stent 10 mounted onto a delivery catheter 11. The stent 10 is a patterned tubular device. The stent 10 typically comprises a plurality of radially expandable, cylindrical rings 12 disposed generally coaxially and interconnected by connectors or links 13 disposed between adjacent cylindrical rings. The delivery catheter 11 has an expandable portion such as a balloon 14 for expanding the stent 10 within an artery 15.

The stent 10 is mounted on a typical delivery catheter 11 used for angioplasty procedures. The balloon 14 may be formed of suitable materials such as polyethylene, polyethylene terephthalate, polyvinyl chloride, nylon and ionomers such as Surlyn®, which is manufactured by the Polymer Products Division of the du Pont Company. Other polymers may also be used. In order for the stent 10 to remain in place on the balloon 14 during delivery to the site of the damage within the artery 15, the stent 10 is compressed or crimped onto the balloon. The crimping can be done mechanically or by hand, at any time prior to delivery of the stent.

Each expandable cylindrical ring 12 of stent 10 may be independently expanded. Therefore, the balloon 14 may be provided with an inflated shape other than cylindrical, e.g., tapered, to facilitate implantation of the stent 10 in a variety of lumen shapes.

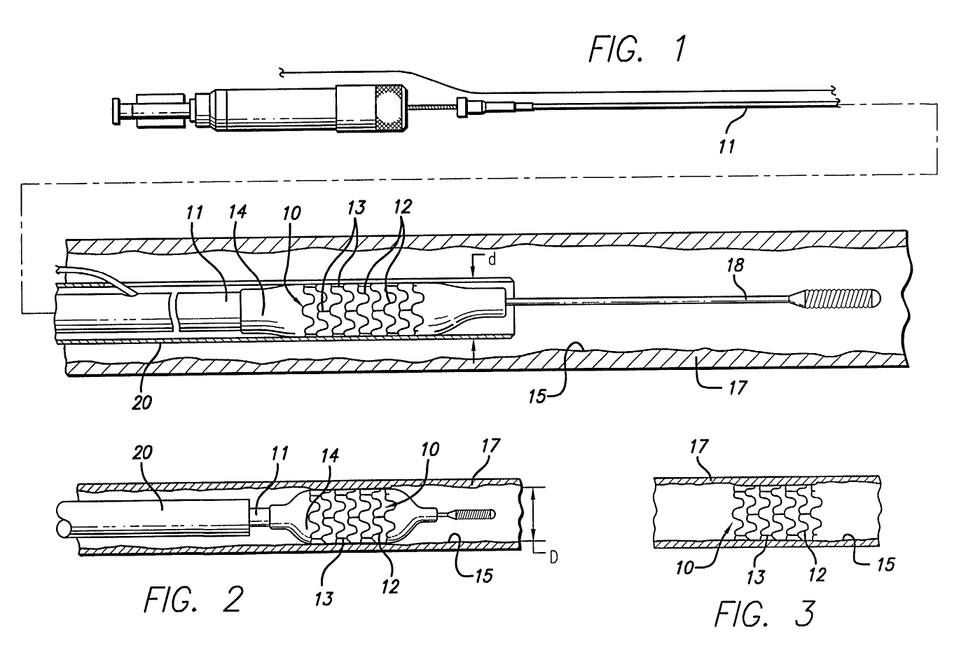

The delivery of stent 10 is accomplished in the following manner. The stent is first mounted onto inflatable balloon 14 on the distal extremity of delivery catheter 11. For the present invention, crimping would be used. Balloon 14 is slightly inflated to secure the stent 10 onto the exterior of the balloon. It is anticipated that the present invention will lessen the degree of inflation necessary, or perhaps even eliminate it. Thus, the present invention will reduce the delivery diameter “d” of stent 10 even farther, thus enhancing the stent's deliverability. The catheter-stent assembly is introduced into the patient's vasculature by a conventional Seldinger technique through a guiding catheter (not shown). Guide wire 18 is disposed across the desired arterial section and then the catheter-stent assembly is advanced over guide wire 18 within the artery 15 until the stent 10 is also located in the desired arterial section. The catheter balloon 14 expands the stent 10 against the artery 15 to expanded diameter “D”, as illustrated in FIG. 2. In practice, artery 15 is often overexpanded slightly by the expansion of stent 10 to fix stent 10 to avoid loosening and migration.

Stent 10 holds open artery 15 after balloon 14 is deflated catheter 11 is withdrawn, as illustrated by FIG. 3. Because stent 10 is formed from a thin-walled, elongated tubular member, the undulating components of the cylindrical rings are relatively flat in transverse cross-section. Thus, when stent 10 is expanded, the rings 12 are pressed into the wall of artery 15 and do not interfere with the blood flow through artery 15. The expanded rings 12 will eventually be covered with endothelial cell growth, which further minimizes blood flow interference. The undulating portion of the cylindrical sections 12 provide good tacking characteristics to prevent stent movement within the artery. Furthermore, the closely spaced rings 12 also provide uniform support for the wall of the artery 15, and consequently are well adapted to tack up and hold in place stenosis, small flaps, or dissections in the wall of the artery 15.

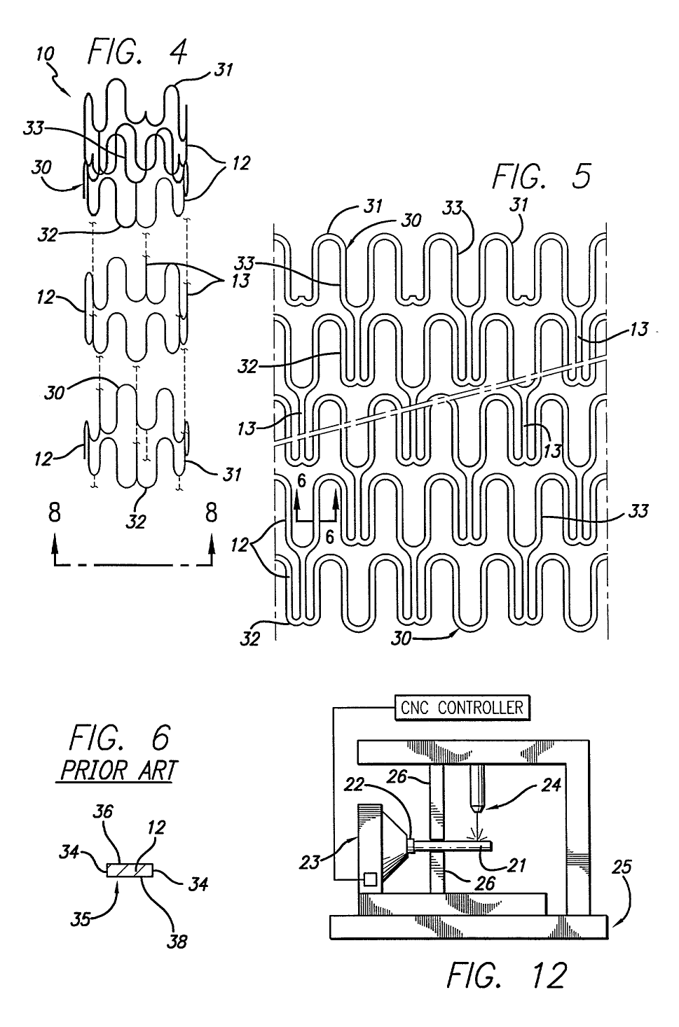

FIG. 4 is a perspective view of the stent 10 shown in FIG. 1. One end of the stent is shown in an exploded view to illustrate in greater detail the placement of links 13 between adjacent radially expandable rings 12. Each set of the links on one side of a ring are preferably placed to achieve maximum flexibility for a stent. In the embodiment shown in FIG. 4, the stent has three links between adjacent radially expandable rings. The links are spaced 120° apart. Each set of links on one side of a ring is offset circumferentially 60° from the set on the other side of the ring. The alternation of the links results in a stent which is longitudinally flexible in essentially all directions. While the straight links themselves do not provide flexibility to the stent, which is provided primarily by the rings, the positioning of the links allows the stent to bend in any direction. Various configurations for the placement of links are possible. For example, the links may have bends or curves to enhance stent flexibility. However, as previously mentioned, it is preferred that all of the links of an individual stent should be secured to either the peaks or valleys of the undulating to prevent shortening of the stent during expansion.

As best observed in FIGS. 4 and 5, rings 12 are in the form of a serpentine pattern 30. While the preferred embodiment of stent 10 does not have discrete parts, for ease of discussion it can be described as having a serpentine pattern 30 made up of a plurality of U-shaped members 31, W-shaped members 32, and Y-shaped members 33, each having bends that have a different radius of curvature so that expansion forces are more evenly distributed over the various members.

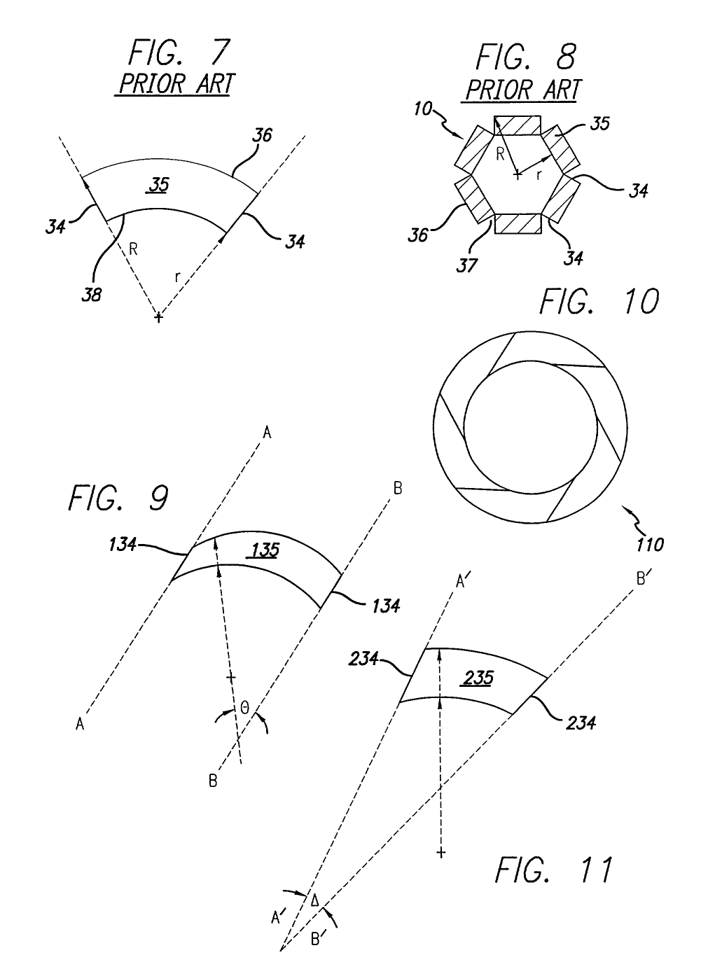

FIGS. 6 and 7 represent a cross-section of a strut 35 from ring 12 in FIG. 5. The strut 35 has two sides or edges 34 and top and bottom surfaces 36 and 38 respectively. Top surface 36 is part of the outer surface of stent 10. Bottom surface 36 forms the interior of the sent. Those in the art typically refer to the cross-sectional shape of struts as rectangular. FIG. 6 depicts such a shape. When stents are cut from a solid tube, however, the struts have an arcuate shape as depicted in FIG. 7. Nevertheless, the struts are still considered generally rectangular by those in the art, because the angle of the arc traversed by the strut surface is relatively small and the stent edges are almost parallel. For example, FIG. 7 exaggerates the arc traversed by a typical strut, solely for the purpose of clarity and explanation of the actual shape. A stent strut 35 will typically look more like FIG. 6 or FIG. 8 than FIG. 7.

FIG. 8 conceptually depicts a cross-section of a crimped prior art stent 10. Although the stent in FIG. 8 is not perfectly circular, it can still be thought of as such. Thus, “+” is the center of stent 10, with an outside radius “R” and inside radius “r.” Because the cross-sections of struts 35 are, however, generally rectangular, when crimped they leave spaces 37 between the edges 34 of crimped stent 10. Eliminating spaces 37 results in a stent with a smaller outside radius, and therefore lowers the stent profile for delivery into a patient's vascular system.

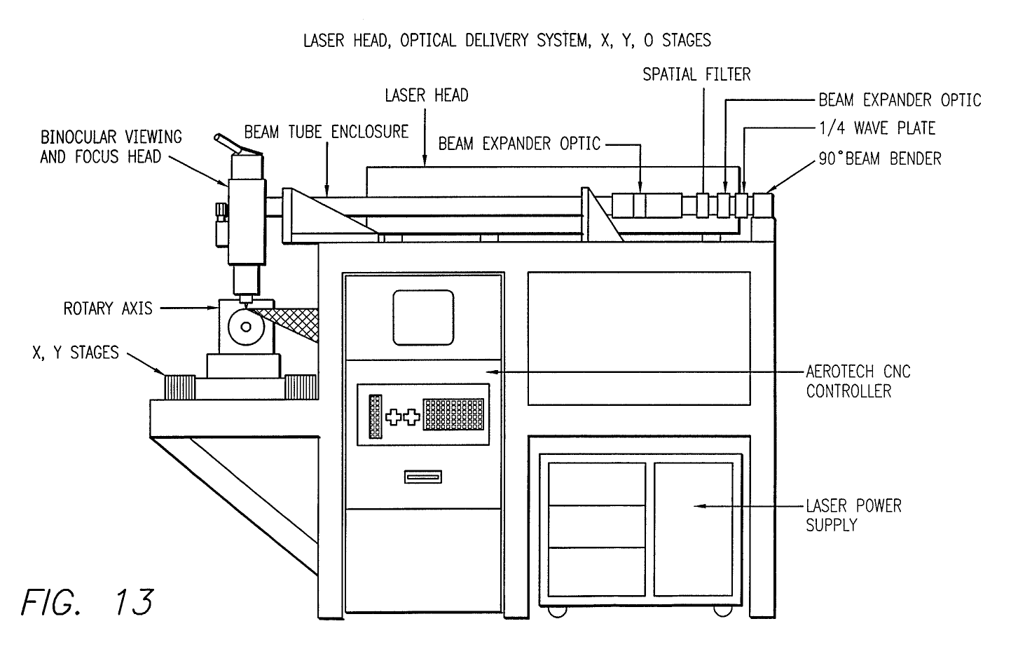

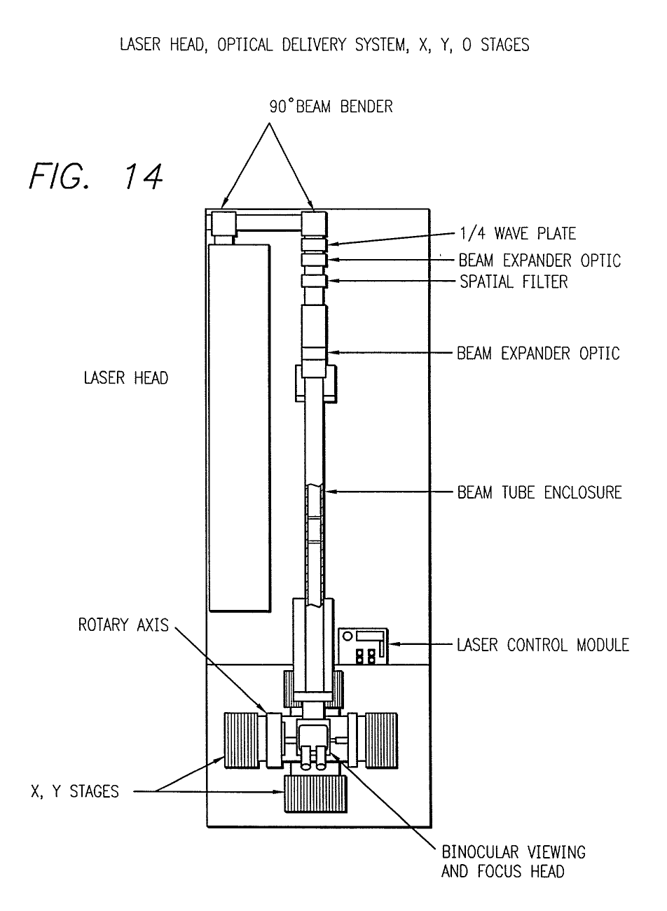

FIG. 9 depicts a strut 135 that permits such a lower profile, resulting in a crimped stent that looks more like FIG. 10 than FIG. 8. The edges 134 of strut 135 are cut parallel to each other, as indicated by parallel lines A-A and B-B that are colinear with edges 134. As a result, the lines defined by strut edges 134 form an angle θ with the stent radius. In contrast, prior art stents, with struts 35 like that in FIG. 7, had strut edges 34 that did not form an angle with the stent radius, but rather were colinear with it.

FIG. 11 shows a slight modification of the present invention. Strut 235 has edges 234 that define lines A′A′ and B′-B′. While lines A′-A′ and B′-B′ are not parallel, they are close to parallel, forming an acute angle “Δ” at a distance far greater from the stent's surface than the stent's radius. Another way of differentiating struts 135 and 235 from prior art struts is in how edges 134 and 234 are cut. Edges 134 are all cut in the same direction, as are edges 234. In contrast edges 34 in FIGS. 6 and 7 can be described as cut in opposite directions. Thus, a stent with struts 135 or 235 will, when crimped, create a substantially tessellated surface, resulting in a configuration more like stent 110, in FIG. 10 which will ideally have a lower profile than its prior art counterpart in FIGS. 4-7. Depending up how the stent pattern is configured, the entire stent surface may not be completely tessellated. If the stent's surface is substantially tessellated, it should still provide the advantages of the present invention.

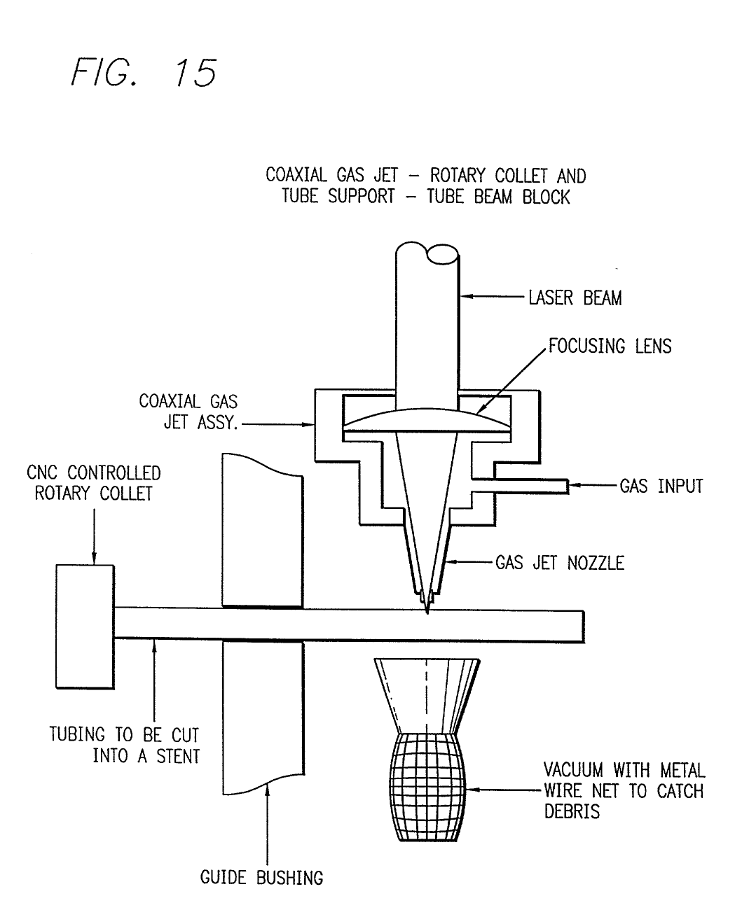

The stent 10 can be any of any configuration and can be made in many ways. However, the preferred method of making the stent is to cut thin-walled stainless steel tubing and to remove portions of the tubing in the desired pattern for the stent, leaving relatively untouched the portions of the metallic tubing which form the stent. The stainless steel is preferably 316L or 316L SS. The preferred method of making the stent requires cutting the tubing in the desired pattern by means of a machine-controlled laser as illustrated schematically in FIG. 12.

As mentioned above, the tubing may be made of suitable biocompatible material such as stainless steel. Cobalt-chromium or platinum-modified stainless steel may also be used. There is a significant difference between stainless steel stent cutting and cobalt-chromium and platinum-modified stainless steel stent cutting. Higher peak power, non-reactive assistant gas (dry air instead of oxygen) and smaller gas jet stand off ((0.015 inch (0.0381 mm) instead of 0.025 inch (0.0635 mm)) need to be set for cobalt-chromium and platinum-modified stent cutting.

Referring to FIG. 12, the tubing 21 is put in a rotatable collet fixture 22 of a CNC-controlled rotary apparatus 23. The tubing 21 is positioned relative to a laser 24. According to machine-encoded instructions, the tubing 21 is rotated and moved longitudinally relative to the laser 24, which is also machine-controlled. The laser selectively removes the material from the tubing by ablation and cuts a predetermined pattern into the tube. The program for control of the apparatus is dependent on the particular machine configuration used and the stent's pattern.

The important step in the present method occurs in aligning the laser beam. While past methods would align the beam along the stent's radius, the present method requires that the laser is offset from the stent's center, so it does not cut along the stent's radius. The method can also be adapted to a manufacturing process in which the stent is formed from a flat piece of material which is cut and then rolled into a tubular shape and welded.

Referring now to FIGS. 13-15 of the drawings, there is shown a process and apparatus, in accordance with the invention, for producing metal stents with a fine precision structure cut from a small diameter thin-walled cylindrical tube. Cutting a fine structure (e.g., 0.0035 inch web width (0.0889 mm)) requires minimal heat input and the ability to manipulate the tube with precision. It is also necessary to support the tube yet not allow the stent structure to distort during the cutting operation. In order to successfully achieve the desired end results, the entire system must be configured very carefully. The tubes are made of stainless steel with an outside diameter in the range of 0.050 inch to 0.080 inch (1.27 to 2.032 mm) and a wall thickness in the range of 0.002 inch to 0.008 inch (0.0508 to 0.2032 mm). The stainless steel can be 316L, 316L SS; it can be modified by cobalt chromium or platinum; or it can be any material approved for surgical implants. These tubes are fixed on the collet under a laser and positioned utilizing a CNC device. Due to the thin wall and the small geometry of the stent pattern (0.0035 inch typical web width), it is necessary to have very precise control of the laser, its power level, the focused spot size, and the precise positioning of the laser cutting path.

In order to minimize the heat input into the stent structure, which prevents thermal distortion, uncontrolled burn out of the metal, and metallurgical damage due to excessive heat, and thereby produce a smooth, cut, the stent is cut finely by a focused laser beam (in the range of 0.0005 inch to 0.0008 inch spot size (0.0127 to 0.02032 mm)) through the coaxial gas jet (nozzle) on the working surface while the tubing is precisely controlled by the CNC system. A pulsed Nd:YAG laser, such as that offered by Lasag Industrial Lasers, is used here. Approximately 0.015 inch (0.381 mm) spacing (nozzle stand-off) is set between the tip of the gas jet and the tubing surface.

With the present system, it is possible to make smooth, narrow cuts with very fine geometries without damaging the narrow webs or struts that make up the stent structure. Hence, the present invention makes it possible to adjust the laser parameters to cut narrow kerf width which will minimize the heat input into the material.

Alternate manufacturing conditions may present a situation whereby other lasers may be used, such as a Q-switched YAG laser or a diode-pumped YAY laser. Other non-reactive gases, like helium, argon or nitrogen are also permissible alternatives.

The CNC equipment is manufactured and sold by Aerotech Corporation. It has a unique rotary mechanism that allows the computer program to be written as if the pattern were being cut from a flat sheet. This allows both circular and linear interpolation in the programming. Since the stent is very small, a precision drive mechanism is required.

The optical system which expands the original laser beam delivers the beam through a viewing head and focuses the beam onto the surface of the tube. The system incorporates a coaxial gas jet and nozzle that helps to remove debris from the kerf and cool the region where the beam interacts with the vaporizing metal.

An optional second tube can be placed inside the stent tube which has an opening to trap the excess energy in the beam which is transmitted through the kerf along with collecting the debris that is ejected from the laser cut kerf. A vacuum or positive pressure can be placed in this shielding tube to remove the collection of debris.

In most cases, the gas utilized in the jets is non-reactive (inert) dry air. Compressed dry air offers more control of the material removed and reduces the thermal effects of the material itself. Gasses such as argon, helium, or nitrogen can be used to prevent any oxidation of the cut material. There is, however usually a tail of molten material that collects along the exit side of the gas jet that must be mechanically or chemically removed after the cutting operation.

The cutting process results in a very narrow kerf (in the range of about 0.0005 inch to 0.0008 inch (0.0127 to 0.02032 mm)), with the molten slag re-solidifying along the cut. This traps the cut out scrap and requires further processing. In order to remove the slag from the cut, it is necessary to soak the cut tube in a solution of HCL for approximately 8 minutes at a temperature of approximately 55° C. (131° F.). Before it is soaked, the tube is placed in a bath of alcohol and water and ultrasonically cleaned for approximately 1 minute. This process removes the loose debris left from the cutting operation. After soaking, the tube is then ultrasonically cleaned in the heated HCL for 1-4 minutes, depending upon the wall thickness. To prevent cracking or breaking of the struts at the two ends of the stent due to the harmonic oscillations induced by the ultrasonic cleaner, a mandrel is placed down the center of the tube during the cleaning and scrap removal process. At the completion of this process, the stent structures are rinsed in water.

The next step is electropolishing. Descaling yields a roughened but clean surface. Stents, being relatively small and fragile, are well suited to electropolishing, but not to grinding, vibration, or tumbling to attain a smooth finish.

Sometimes referred to as “reverse plating,” the process of electropolishing actually removes metal from the surface. Electropolishing is an electrochemical process that smooths metal surfaces by dissolution of metal, which takes place more rapidly at high points on the metal surface. The metal stent is rendered anodic (+) and is immersed in a liquid electrolytic solution along with a metal cathode (−). Current is applied and flows from the anode, polarizing it, and causing the metal ions to diffuse through the solution to the cathode.

A special feature of electropolishing is the creation of current differentials across the microscopic surface of the anode. The current density is greatest at high points on the surface and lowest at the low points. The rate of the electrochemical reaction is directly proportional to the current density, so that increased current density at the raised points causes the anodic metal to dissolve faster at these points, thus leveling the surface material. The smoothed surface of many metals can, with sufficient electropolishing techniques including the use of the proper electrolytic solution, be made so smooth that they become shiny and reflective. The finish may also be dependant on the level of current applied, the duration of applied current, and the temperature of the electrolytic solution.

The stents are electrochemically polished in an acidic aqueous solution such as a solution of ELECTRO-GLO #300, sold by the ELECTRO-GLO Co., Inc,. Chicago, Ill. The mixture includes sulfuric acid, carboxylic acid, phosphates, corrosion inhibitors and a biodegradable surface active agent. The bath temperature is maintained at about 110-135° F. (43.3° C. to 57.2° C.) and the current density is about 0.4 to about 1.5 amps per in.² Minimum cathode to anode area should be about two to one. The stents may be further treated if desired, for example by applying a biocompatible coating. Other descaling and electropolishing processes are well known in the art and are especially suitable to finishing and smoothing the laser cut stent.

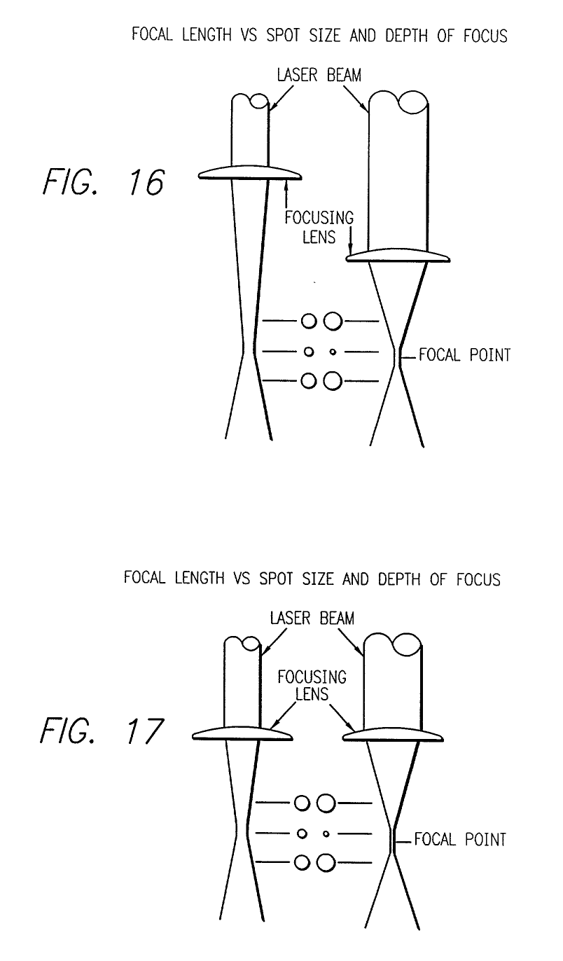

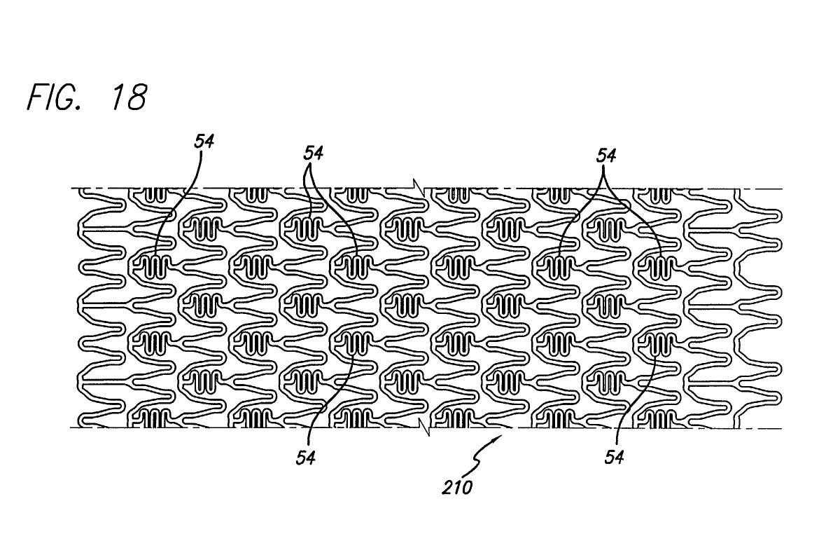

Referring now more particularly to FIGS. 16 and 17, it will be apparent that both focused laser spot size and depth of focus can be controlled by selecting beam diameter (FIG. 16) and focal length for the focusing lens (FIG. 17). It will be apparent from FIGS. 16 and 17 that increasing laser beam diameter, or reducing lens focal length, reduces spot size at the cost of depth of field.

It will be apparent from the foregoing that, while particular forms of the invention have been illustrated and described, various modifications can be made without departing from the spirit and scope of the invention. Accordingly, it is not intended that the invention be limited, except as by the appended claims. For example, in one embodiment (not shown) of the invention, one ring has narrower struts than the struts of adjacent ring elements. The ring elements with narrower struts alternate with ring elements having wider struts to provide for maximum grip when the stent is crimped to the delivery catheter yet maintains high radial strength. One advantage of utilizing ring elements with narrower struts is that these narrower struts can be collapsed down to a lower diameter without having struts overlap when the stent is crimped. This configuration augments the features of a stent with struts that can be created with a tessellated surface. Another embodiment of the invention includes ring elements which have narrower struts and also shorter axial lengths to help improve the composite radial strength of the stent. The use of alternating wider and narrower struts still maintains high radial strength, radiopacity, and sufficient coverage to adequately open and support the wall of the body lumen. Another embodiment of the invention contemplates improved flexible links 54 of stent pattern 210, as depicted in FIG. 18.

What is claimed:

1. An endoluminal prosthesis, comprising:
a stent having a substantially tessellated surface.

2. The prosthesis of claim 1, wherein the stent comprises a plurality of connected rings.

3. The prosthesis of claim 2, wherein the rings are undulating.

4. The prosthesis of claim 3, wherein the rings comprise struts with a non-rectangular, parallelogram cross-section.

5. An expandable stent for use in a body lumen, comprising:
a plurality of adjacent, interconnected, expandable rings, each ring having a first, uncrimped diameter and a second, crimped diameter;
at least one connector member connecting each adjacent pair of rings; and
wherein the rings form a tube with a substantially tessellated surface when the rings are in the second, crimped diameter.

6. The stent of claim 5, wherein the rings and connector members comprise a plurality of apertures and a generally continuous material defining a wall surface of the tube.

7. The stent of claim 6, wherein the material has a generally non-rectangular, parallelogram cross-section.

8. The stent of claim 7, wherein the parallelogram has a top that forms the tessellated surface and two sides disposed angularly to a radius defined by a tube center and the tube surface.

9. In an expandable, crimpable, metal stent having interconnected, undulating rings comprised of struts, the improvement comprising:
struts with a generally non-rectangular, parallelogram cross-section that form a substantially tessellated stent surface when crimped.

10. In a crimped stent comprising a plurality of interconnected rings that form a tubularly shaped metal lattice, the improvement comprising:
a substantially tessellated tube surface defined by generally abutting lattice portions having parallel edges angularly disposed to a tube radius and having parallel top and bottom surfaces defining an inner and outer surface of the stent.

11. A method of making an expandable stent having a reduced profile when it is crimped, comprising:
providing a generally tubular section with an inner and outer surface and a tube radius;
supporting the tubular section for computer controlled motion relative to a stent cutter;
aligning the stent cutter angularly to the tube radius, so that cuts through the tube are not colinear with the tube radius; and
cutting a precise pattern into the tubing to form the stent.

12. The method of claim 11, wherein the tubular section has an uncrimped diameter equal to or larger than the length of the tube.

13. The method of claim 12, wherein the stent comprises a plurality of connecting tubular sections.

14. The method of claim 11, wherein the tube cutter is a laser.

15. The method of claim 11, wherein the tube cutter is an electrical discharge machine.

16. The method of claim 11, wherein a flat material is provided, cut as claimed, and then formed into the tubular section.

17. A method of making an expandable, crimpable metal stent, comprising:
providing a metal, generally tubular section with a center and with an inner and outer surface defining a tube thickness colinear with a tube radius;
supporting the tubular section for computer controlled motion;
aligning a laser beam on the outer surface of the tubular section so that the laser beam is not colinear with the tubular section's center; and
cutting a precise pattern into the tubular section to form the stent, wherein the pattern includes adjacent cross-sections of metal with parallel edges that abut along their length when crimped.

18. The method of claim 17, wherein a pulsed YAG laser impinges the laser beam on the working surface of the metal tube.

19. The method of claim 17, wherein a stream of pressurized air is directed through a coaxial jet nozzle toward the metal tube to cool and remove debris from the tubing.

20. The method of claim 17, wherein the laser beam is circularly polarized.

21. The method of claim 20, wherein the circular polarization is accomplished by a quarter wave plate.

22. The method of claim 17, wherein the laser beam is spatially filtered.

23. The method of claim 17, wherein the size of the focused laser beam spot and depth of field is controlled by selecting a beam diameter.

24. The method of claim 23, wherein selecting a focal length of the beam focusing lens controls the size of the focused laser beam spot and depth of field.

25. The method of claim 17, wherein the laser beam passes through a coaxial gas stream adjacent the metal tube.
